(19) United States
(12) Patent Application Publication
(10) Pub. No.: US 2026/0216078 A1
(43) Pub. Date: Jul. 30, 2026

(54) WATER-SOLUBLE CAPSAICIN METAL COMPLEXES AND METHOD OF PREPARATION THEREOF

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/396,267

(22) Filed: Nov. 20, 2025

Related U.S. Application Data
(60) Provisional application No. 63/722,869, filed on Nov. 20, 2024.

(51) Int. Cl. A61K 9/14; A61K 9/00; A61K 31/166

(52) U.S. Cl. CPC A61K 9/14; A61K 9/0053; A61K 31/166

(57) ABSTRACT

A metal-capsaicinoid complex that renders the capsaicinoid water-soluble is disclosed along with related compositions. Methods of producing the metal-capsaicinoid complex are also disclosed.

### CROSS-REFERENCE TO RELATED APPLICATION

This application claims priority to and the benefit of U.S. Provisional Patent Application 63/722,869 filed Nov. 20, 2024 and titled “WATER-SOLUBLE CAPSAICIN METAL COMPLEXES AND METHODS OF PREPARATION THEREOF”, the entire contents of which are hereby incorporated herein by reference.

### TECHNICAL FIELD

This disclosure relates to the development of water-soluble forms of capsaicin and their use in pharmaceutical and nutraceutical formulation.

### BACKGROUND

Capsaicinoids are a group of alkaloid compounds responsible for the spicy, pungent taste of hot chili peppers, which are plants belonging to the genus Capsicum. It is thought that capsaicinoids are produced as secondary metabolites by chili peppers as deterrents against certain mammals and fungi. The family of capsaicinoids include capsaicin, dihydrocapsaicin, capsaicinol capsiate, and nonivamide.

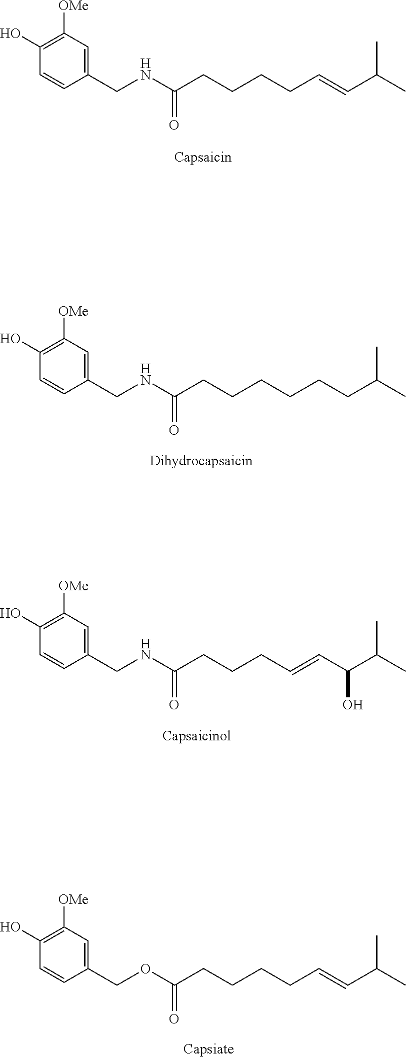 [SMILES: COc1cc(CNC(=O)CCCC/C=C/C(C)C)ccc1O ; COc1cc(CNC(=O)CCCCCCC(C)C)ccc1O ; COc1cc(CNC(=O)CCC/C=C/[C@H](O)C(C)C)ccc1O ; COc1cc(COC(=O)CCCC/C=C/C(C)C)ccc1O]

Capsaicin (8-methyl-N-vanillyl-6-nonenamide) is a potent irritant for mammals, including humans, and produces a sensation of burning in any tissue with which it comes into contact. Capsaicin is used to provide pain relief, as an active ingredient in anti-inflammatory products, and to enhance metabolism. However, the hydrophobicity of capsaicin limits its bioavailability and efficacy when used in per os compositions and formulations prepared in an aqueous environment, such as, a beverage, an aqueous-based topical composition, or an injectable product. Capsaicinoids that have similar pharmacological effects as capsaicin are dihydrocapsaicin, capsaicinol, capsiate, and nonivamide, but these capsaicinoids have similar bioavailability issues as capsaicin. Thus, there is a long felt need for a novel method to render capsaicin water-soluble without losing its effectiveness or desired biological properties.

Capsaicin is used to provide pain relief, as an active ingredient in anti-inflammatory products, and to enhance metabolism. However, the hydrophobicity of capsaicin limits its bioavailability and efficacy when used in per os compositions and formulations prepared in an aqueous environment, such as, a beverage, an aqueous-based topical composition, or an injectable product. Capsaicinoids that have similar pharmacological effects as capsaicin are dihydrocapsaicin, capsaicinol, capsiate, and nonivamide, but these capsaicinoids have similar bioavailability issues as capsaicin.

Thus, there is a long felt need for a novel method to render capsaicin water-soluble without losing its effectiveness or desired biological properties.

### SUMMARY

The present invention discloses a method for making capsaicinoids water-soluble. In other words, a method of producing a water-soluble form of a capsaicinoid is disclosed. The method produces a metal-capsaicinoid complex that is water-soluble by reacting the capsaicinoid with an alkali metal component (such as metallic lithium, metallic sodium, metallic potassium or their various alkoxides, including the strong bases KOH, LiOH, and NaOH). The produced metal-capsaicinoid complex has increased solubility of the capsaicinoid in aqueous solutions while enhancing its bioactivity. For example, the metal-capsaicinoid complex has a solubility of at least 0.1 g/100 ml in water. This water-soluble metal-capsaicinoid complex can be used in a variety of pharmaceutical, nutraceutical, dietary supplement and cosmetic applications.

The invention also includes a method of producing the water-soluble metal-capsaicinoid complexes. In a particular implementation, the capsaicinoid is solubilized in a polar, preferably anhydrous, solvent containing in solution the metal component and then drying out the solution.

In certain implementations, the method of producing the metal-capsaicinoid complex comprises dissolving the metal component in a polar solvent to produce a solution comprising the dissolved metal base, and then dissolving the capsaicinoid in the solution comprising the dissolved metal base to produce a complexing solution. The complexing solution is stirred until a homogenous solution is formed, and then the polar solvent in the homogenous solution is evaporated, thereby producing a dried product comprising metal-capsaicinoid complex.

Also disclosed herein are compositions comprising the water-soluble form of capsaicinoid. For example, a composition may comprise 5-95% by weight of a capsaicinoid and 5-95% by weight of a metal. The composition may also comprise by 5-95% a metal alkoxide, such as potassium ethoxide and sodium ethoxide. At least a portion of the capsaicinoid is complexed to the metal and/or its alkoxide. In some aspects, the capsaicinoid in the composition is not aggregated together while in a solvent. In other implementations, the composition consists of the metal-capsaicinoid complex.

The foregoing and other aspects, features, and advantages will be apparent from the DRAWINGS, DETAILED DESCRIPTION and CLAIMS.

### BRIEF DESCRIPTION OF DRAWINGS

The patent or application file contains at least one drawing executed in color. Copies of this patent or patent application publication with color drawings will be provided by the Office upon request and payment of the necessary fee.

The accompanying drawings are incorporated in and constitute a part of this disclosure, illustrate and provide a further understanding of implementations of the present disclosure, and together with the DETAILED DESCRIPTION serve to explain the principles of the present disclosure.

FIG. 1 shows a capsaicin-potassium complex formed from the preparation method of Example 1.

FIGS. 2-4 show Fourier transform infrared spectroscopy confirming the capsaicin-potassium complex of Example 1.

FIGS. 5-6 show solubility and dissolution of the capsaicin-potassium complex of Example 1.

FIGS. 7-10 show the solubility of capsaicin in an alkaline medium, water, and ethanol, respectively.

FIG. 11 shows a nonivamide-potassium complex formed from the preparation method of Example 5.

FIG. 12 shows Fourier transform infrared spectroscopy confirming the nonivamide-potassium complex of Example 5.

FIG. 13 shows Fourier transform infrared spectrum of nonivamide from Logrado et al., “Counterfeit Pepper Spray: A Case Report.”, Human Factors and Mechanical Engineering for Defense and Safety, 2022, 6: Article 16.

Corresponding reference characters indicate corresponding parts throughout the several views of the figures. The figures represent an illustration of some of the implementations of the present disclosure and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to employ the present invention.

### DETAILED DESCRIPTION

Detailed aspects and applications of the disclosure are described and shown below. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. “About”, as disclosed in this patent will be understood to mean a variation from the disclosed numbers between 1-2%, 1-5% or even 1-20%. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, “pharmaceutically acceptable additive” or “additive” are terms used in their broadest sense. Particular implementations of the compositions described in this document may also comprise an additive (e.g. one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically effective additives from the disclosure in this document. In particular implementations, pharmaceutically acceptable additives may include, by non-limiting examples, calcium phosphate, cellulose, stearic acid, crosscarmelose cellulose, magnesium stearate, and silicon dioxide.

As used in this document, “pharmaceutically effective” is a phrase used in its broadest sense, including, by non-limiting example, effective in a clinical trial or for a specific patient. When used in a method claim, pharmaceutically effective will mean in a dose enough to achieve the claim's preamble.

As used in this document, “pharmaceutically acceptable” is a phrase used in its broadest sense and may describe ingredients of a pharmaceutical composition that meet Food and Drug Administration (FDA) standards, United States Pharmacopeial Standards (USP), US Department of Agriculture (USDA) standards for food-grade materials, commonly accepted standards of the nutritional supplement industry, industry standards, botanical standards, or standards established by any individual. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

As used herein, the term “disease” refers to a state of health of an animal (including humans) wherein the animal cannot maintain homeostasis, and wherein if the disease is not ameliorated then the animal's health continues to deteriorate.

In contrast, as used herein, the term “disorder” in an animal refers to a state of health in which the animal (including humans) is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

A disease is “cured” if the disease no longer shows any symptoms and appropriate diagnostic tests show absence of it on a patient.

As used herein, “treat,” “treatment,” and “treating,” a disease or disorder means reducing the severity and/or frequency, with which a sign and/or symptom of the disease or disorder is experienced by a subject. The subject may be symptomatic or asymptomatic at the time of treatment. In other words, “treat,” “treatment,” and “treating,” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

It will be understood that the term “capsaicin” refers to the molecule of capsaicin itself (which can be isolated from plants such as peppers or synthetically produced) along with its metabolites or derivatives such as salts and esters. The term “capsaicinoids” refers to well documented and understood group of compounds that are chemically similar to capsaicin and produce similar biological effects, such as the activation of the vanilloid type 1 (TRPV1) receptor. The general formula of capsaicinoids is shown below along with the unique R groups that differentiate the capsaicinoid compounds.

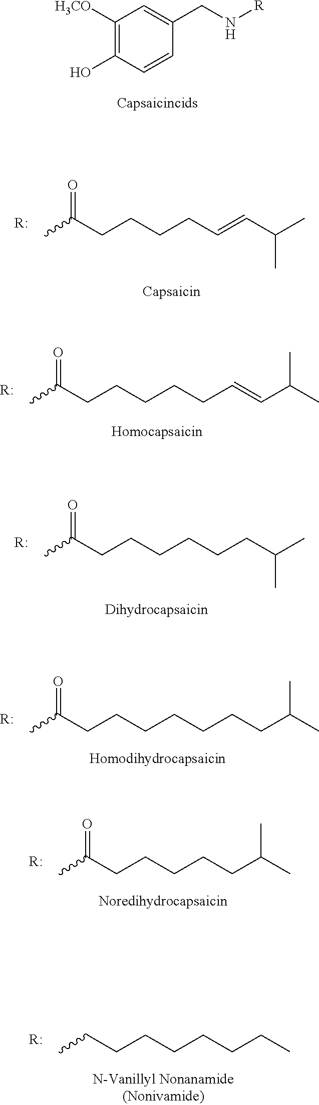

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions.

Disclosed herein are methods of producing water-soluble forms of a capsaicinoid, which provide improved bioavailability, solubility, versatility, and practicability of the capsaicinoid for use in various applications, including topical, oral, and injectable formulations. Compounds produced by the methods described herein shall be referred to as water-soluble metal-capsaicinoid complexes. It will be understood that for purposes of this's disclosure the order of the metal and capsaicinoid in the naming of the complex is immaterial. For example, “sodium-capsaicin complex” and “capsaicin-sodium complex” are the same thing. Also, unless otherwise specified, the term “metal” shall refer to one of sodium, lithium, and potassium.

In spite of the potential benefits that can be derived from oral administration of capsaicinoids, this route of administration is limited by their low water solubility. Capsaicin is practically insoluble in water (0.0013 g/100 mL) which creates challenges in formulations, especially when fats are not desired, such as in water-based gels serums and creams while also severely limiting its oral bioavailability. The metabolites of capsaicin in the body are as follows:

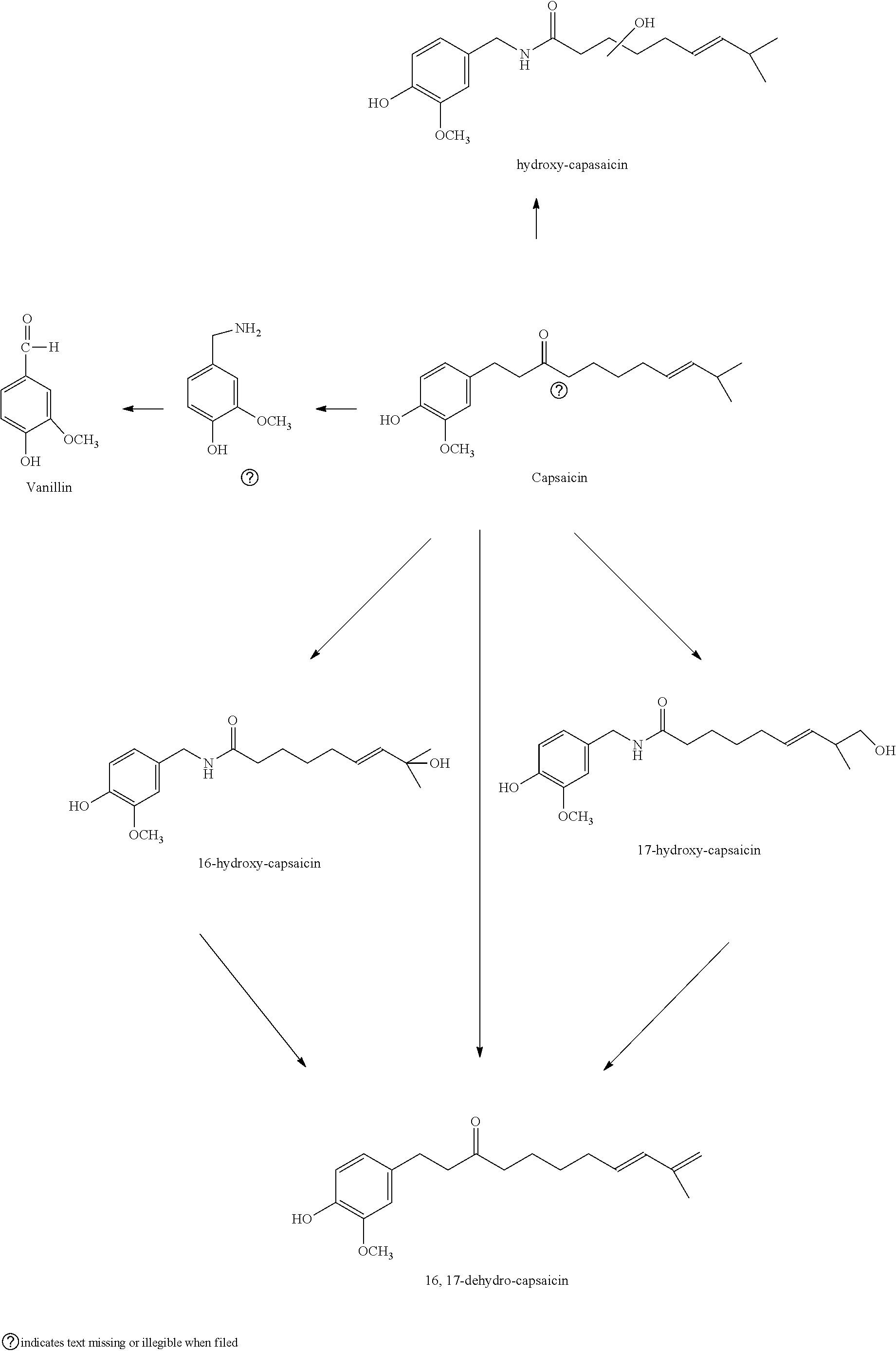

It has been described that (O'Neill J, Brock C, Olesen A E, Andresen T, Nilsson M, Dickenson A H. “Unravelling the mystery of capsaicin: a tool to understand and treat pain.” Pharmacol Rev., 2012, 64 (4): 939-71):
The literature of orally administered capsaicin in humans is sparse. A recent study investigated thoroughly the human pharmacokinetic profile of 5 g of orally ingested C. frutescens, equipotent to 26.6 mg of pure capsaicin (this is the equivalent of eating 15 habanero peppers!) (Chaiyasit et al., “Pharmacokinetic and the effect of capsaicin in Capsicum frutescens on decreasing plasma glucose level.” J Med Assoc Thai, 2009, 92:108-113). The group documented that capsaicin could be detected in plasma after 10 min, and that peak concentration (Cₘₐₓ) was 2.47±0.13 ng/ml, tₘₐₓ was 47.1±2.0 min, and t_{1/2} was 24.9±5.0 min (Chaiyasit et al., 2009). After 90 min, capsaicin could not be detected (Chaiyasit et al., 2009). These results suggest that Bernard et al. were unable to create a pharmacokinetic profile of capsaicinoids after administration of 15 and 30 mg of capsaicin/person, because they used a lower detection limit of 10 ng/ml (Bernard et al., “Studies of the toxicological potential of capsinoids: X. Safety assessment and pharmacokinetics of capsinoids in healthy male volunteers after a single oral ingestion of CH-19 Sweet extract.” Int J Toxicol, 2008, 27 (Suppl 3): 137-147).

Current research efforts to enhance the oral bioavailability of capsaicin have been focused predominantly on the nanoencapsulation strategy motivated by the prolonged capsaicin retention time afforded by nanocapsules (Tran and Hadinoto, “A new solubility enhancement strategy of capsaicin in the form of high-payload submicron capsaicin-chitosan colloidal complex,” Colloids and Surfaces A: Physicochemical and Engineering Aspects, 2017. 520:62-71). To the best of the inventors' knowledge, no commercially available water-soluble form of capsaicin exists. There is therefore a long felt need in the art for readily water-soluble capsaicin forms.

The inventors addressed this problem by producing, for the first time, a water-soluble form of capsaicinoids. Disclosed is a novel complex of a capsaicinoid with a metal, for example an alkaline metal. Thus, in some aspects, the water-soluble form of a capsaicinoid such as capsaicin forms a complex with a strong base such as KOH, NaOH, and LiOH. The method of producing the water-soluble form of capsaicinoids comprises first dissolving a metal component (such as an alkaline metal hydroxide) in a polar solvent and then dissolving the capsaicinoid in the solution containing the dissolved metal component.

The polar solvent may be selected from, by non-limiting example, methanol, ethanol, propanol, butanol, isopropyl alcohol, dichloromethane, and dimethyl sulfoxide (DMSO). The polar solvent preferably contains low amounts of water, as high water content can prevent solubilization of capsaicin and formation of the complex. For example, the polar solvent may be less than 5%, even less than 1%, or even less than 0.1% water. In some aspects, the polar solvent is anhydrous.

In some implementations, the metal component is an alkali metal. In certain embodiments, the metal component is a strong base, and the strong base is selected from the group consisting of LiOH, NaOH, and KOH. In other embodiments, the metal component is the elemental form of the metal component, such as elemental lithium, elemental potassium, or elemental sodium. In other implementations, the metal component is in the form of a hydride, such as Sodium Hydride, Potassium Hydride and Lithium Hydride. In such embodiments, for example, potassium metal can be reacted with ethanol to produce potassium ethoxide, the solution of which can then be used to dissolve the capsaicinoid for the production of the potassium-capsaicinoid complex which can then been dried to produce the metal-capsaicinoid complex. In some implementations, the molar ratio of the capsaicinoid and the metal component used to produce the water-soluble metal-capsaicinoid complex is 1:1, though other ratios with comparatively greater amounts of the metal component can also be used, for example a molar ratio of between one moles capsaicinoid to 1-1000 moles of the metal component, preferably between 1:1-1:100 and more preferably between 1:1-1:10. The metal component is the limiting reagent for producing the water-soluble metal-capsaicinoid complex. Thus, using lower molar ratios of the metal compound will result in an incomplete formation of the metal-capsaicinoid complex. For example, when the molar ratio of the capsaicinoid and the metal component is less than 1:1, for example 0.5:1, not all of the capsaicinoid added to the reaction will form a complex with the metal. As such, only some of the capsaicin will be converted to the water-soluble metal complex, while some will remain in hydrophobic (water-insoluble) form.

As shown in the examples, the complexed capsaicinoid produced in the method is readily water-soluble in water at room temperature or at temperatures as low as −1° C., such as temperature conditions that can be provided in a household refrigerator. Accordingly, in some implementations of the method of producing the water-soluble form of capsaicinoids, the method further comprises isolating the solid (undissolved) portion of the dried product dissolved in water—for example by filtering—and then repeating the steps of producing the water-soluble form of capsaicinoids with the isolated solid portion. As the isolated solid portion comprises non-complexed capsaicinoid, it can be combined with newly produced solution containing a dissolved metal component in order to complex the remaining non-complexed capsaicinoid.

The method of producing the water-soluble form of capsaicinoids further comprises drying the solution containing the dissolved metal component and the capsaicinoid. The resulting product after the drying step comprises the water-soluble metal-capsaicinoid complex. The drying step (evaporation of the solvent) is preferably carried under vacuum or with air drying, since capsaicin is unstable at higher temperatures. However, any conventional method for drying may be applied, such as oven drying, spray drying, rotary evaporation, microwave drying, desiccation and the like. For drying under vacuum or air drying, ethanol is preferred for its ease to evaporate along with its availability and relatively low toxicity.

The complexing of capsaicin (commercially available capsaicin extracted from pepper plants) with a base (in this example KOH) results in a crystalline waxy substance of yellowish/orange color. The capsaicin-metal complex can turn grayish upon exposure to air. The described method of producing a water-soluble form of capsaicin by complexing it with a strong metal base is highly surprising and unexpected, since capsaicin is an alkaloid, i.e. a compound whose solutions have an alkaline pH. Such compounds de facto are less soluble in alkaline solutions. Thus, while it has been described that capsaicin can be soluble in strong HCl solutions, capsaicin is not expected to dissolve in a solution of a strong base. This was verified as can be seen in example 3, where capsaicin was practically insoluble in solutions of KOH, NaOH and LiOH, even after the passage of days.

It will be understood that the above steps can be modified to achieve the same results. For example, two separate solutions of capsaicin and the strong base could be prepared, mixed and then evaporated, or a mixture of solvents instead of a single solvent can be used.

The disclosure also relates to compositions comprising metal capsaicinoid or of their derivatives and metabolites. In some implementations, the composition further comprises a dissolution enhancer, such as sodium carbonate for example. In some other implementations, the water-soluble metal-capsaicinoid complexes or their derivatives and/or metabolites are combined with a dietary ingredient, a food additive, and/or a pharmaceutically acceptable additive.

Supplements and ingredients that may be used in formulas comprising water-soluble metal-capsaicinoid complexes water-soluble capsaicinoid compounds described herein include but are not limited to: 1,3-DMAA (1,3-dimethylamylamine), 1,3-DMBA (1,3-dimethylbutylamine), 1,4-Butanediol, 1,4-DMAA (1,4-dimethylamylamine), 1-Androsterone, 1-Epiandrosterone, 19-nor-DHEA (19-Nordehydroepiandrosterone), 4-Androsterone, 5-deca zol, 5-HTP (5-Hydroxytryptophan), 5aOHP (17beta-(1-ketoethyl)-androstane-3-one), 6-Bromo (6-Bromo-Androstenedione), 7,8-benzoflavone, 7-alpha-hydroxy-DHEA (7-Alpha-Hydroxy-Dehydroepiandrosterone), 7-beta-hydroxy-DHEA (7-Beta-Hydroxy-Dehydroepiandrosterone), 7-Keto-DHEA, 7-Methoxyflavone, Abscess Root, Abuta, Acacia rigidula, Acai, Acerola, Acetyl-L-Carnitine, Ackee, Aconite, Activated Charcoal, Active hexose correlated compound (AHCC), Adenosine, Adrafinil, Adrenal extract, Adrue, Aegeline, African Wild Potato, Agar, Agaricus Mushroom, Agave, Agmatine, Agrimony, Ajuga nipponensis, Albizia julibrissin, Alchemilla, Alder Buckthorn, Aletris, Alfalfa, Algal Oil, Algin, Alkanna, Allspice, Aloe, Alpha-GPC (Alpha Glycerol Phosphoryl Choline), Alpha-Ketoglutarate (AKG), Alpha-Linolenic Acid (ALA), Alpha-Lipoic Acid, Alpine Ragwort, Alpinia, Amaranth, Ambrette, American Adder's Tongue, American Chestnut, American Dogwood, American Elder, American Ginseng, American Hellebore, American Ivy, American Mistletoe, American Pawpaw, American Spikenard, American White Water Lily, Andarine, Andiroba, Andrographis, Androstenediol, Androstenedione, Androstenetrione, Androsterone, Angel's Trumpet, Angelica archangelica, Angostura, Anhydrous Crystalline Maltose, Anise, Annatto, Antineoplastons, Antioxidants, Apoaequorin, Apple, Apple Cider Vinegar, Apple Polyphenols, Apricot, Apricot Kernel, Arabinoxylan, Arenaria rubra, Arimistane, Aristolochia, Arnica, Arrach, Arrowroot, Arsenic, Artemisia herba-alba, Artichoke, Arum, Asafoetida, Asarabacca, Ascophyllum nodosum, Ascorbigen, Ash, Ashitaba, Ashwagandha, Asian Water Plantain, Asparagus, Asparagus Racemosus, Aspartic Acid, Aspen, Astaxanthin, Astragalus, Atlantic Cedar, Atractylodes, Autumn Crocus, Avens, Avocado, Avocado soy unsaponifiables (ASU), Avocado Sugar Extract, Ayahuasca, Babassu, Bach Flower Remedies, Bacillus coagulans, Bacopa, Bael, Baikal Skullcap, Ba Ji Tian, Bamboo, Banaba, Banana, Baobab, Barley, Basil, Bay Leaf, Bayberry, Bear's Garlic, Bee Pollen, Bee Venom, Beer, Beeswax, Beet, Belladonna, Benfotiamine, Benzoin, Berberine, Bergamot, Beta-Carotene, Beta-Cryptoxanthin, Beta-Glucans, Beta-Hydroxybutyrate (BHB), Beta-Methylphenethylamine (BMPEA), Beta-Sitosterol, Betaine Anhydrous, Betaine Hydrochloride, Betel Nut, Beth root, Betony, Bifidobacterium animalis subsp, lactis, Bifidobacterium bifidum, Bifidobacterium breve, Bifidobacterium longum, Bilberry, Biotin, Birch, Bishop's Weed, Bismuth, Bismuth Nitrate, Bistort, Bitter Almond, Bitter Melon, Bitter Milkwort, Bitter Orange, Bitter Yam, Bittersweet Nightshade, Black Alder, Black Bryony, Black Cohosh, Black Currant, Black Haw, Black Hellebore, Black Hoof Mushroom, Black Horehound, Black Mulberry, Black Mustard, Black Nightshade, Black Pepper, Black Psyllium, Black Raspberry, Black Rice, Black Root, Black Seed, Black Tea, Black Walnut, Blackberry, Blackthorn, Blessed Thistle, Blond Psyllium, Bloodroot, Blue Cohosh, Blue Flag, Blue-Green Algae, Blueberry, Bog Bilberry, Bog Labrador Tea, Bogbean, Bois de Rose Oil, Boldo, Boneset, Borage, Boron, Boswellia serrata, Bovine Cartilage, Bovine Colostrum, Boxwood, Branched-chain Amino Acids (BCAA), Breadfruit, Brewer's Yeast, Brickellia, Bridelia, Broccoli, Broccoli Sprout, Bromelain, Brown Rice, Brussels Sprout, Bryonia, Buchu, Buck's-horn Plantain, Buckhorn Plantain, Buckwheat, Bugle, Bugleweed, Bulbine natalensis, Bulbous Buttercup, Bupleurum, Burdock, Burning Bush, Burr Marigold, Butcher's Broom, Butea Superba, Butterbur, Buttercup, Butternut, Butylated hydroxytoluene (BHT), Cabbage, Cade, Caffeic Acid, Caffeine, Cajeput Oil, Calabar Bean, Calabash Chalk, Calamint, Calamus, Calanus Oil, Calcium, Calcium D-Glucarate, Calea zacatechichi, Calendula, California Poppy, Calotropis, Calumba, Camphor, Camu Camu, Canada Balsam, Canadian Fleabane, Canadian Hemp, Canaigre, Cananga Oil, Cannabichromene (CBC), Cannabidiol (CBD), Cannabidivarin (CBDV), Cannabigerol (CBG), Cannabinol (CBN), Cannabis, Canola Oil, Canthaxanthin, Capers, Caprylic Acid, Capsicum, Caralluma, Caraway, Carbon 60 (C60), Cardamom, Cardarine, Carlina, Carnosine, Carob, Carqueja, Carrageenan, Carrot, Cascara Sagrada, Cascarilla, Casein Peptides, Casein Protein, Cashew, Cassava, Cassia auriculata, Cassia Cinnamon, Cassia nomame, Cassie Absolute, Castor Bean, Castoreum, Cat's Claw, Cat's Foot, Catechu, Catnip, Catuaba, Cauliflower, Celery Centaury, Cereus, Cesium, Cetylated Fatty Acids (CFAs), Ceylon Cinnamon, Ceylon Leadwort, Cha de Bugre, Chaga, Chanca Piedra, Chaparral, Chaulmoogra, Cheken, Chelation Therapy Products, Chenopodium Oil, Chervil, Chia, Chickweed, Chicory, Chinese Cucumber, Chinese Mallow, Chinese Prickly Ash, Chirata, Chitosan, Chive, Chlorella, Chlorine Dioxide, Chlorophyll, Chlorophyllin, Chokeberry, Choline, Chondroitin Sulfate, Chromium, Chrysanthemum, Chrysin, Chuchuhuasi, Chymotrypsin, Cilantro, Cinchona, Cissus quadrangularis, Cistanche deserticola, Citicoline, Citric Acid, Citronella oil, Clary Sage, Clay, Clematis recta, Clivers, Clove, Clown's Mustard plant, Clubmoss, Cnidium, Cobalt, Coca, Cocillana, Cocoa, Coconut, Coconut Oil, Coconut Water, Cod Liver Oil, Codonopsis, Coenzyme Q10, Coffee, Coffee Charcoal, Cola Nut, Coleus, Collagen Peptides, Collagen Type I (native), Collagen Type II (native), Collard, Colloidal Minerals, Colloidal Silver, Colocynth, Coltsfoot, Columbine, Combretum micranthum, Comfrey, Common Stonecrop, Condurango, conjugated Linoleic Acid (CLA), Copaiba Balsam, Copper, Coral, Cordyceps, Coriander, Corkwood Tree, Corn Poppy, Corn Silk, Cornflower, Corydalis yanhusuo, Costus, Cotton, Couch Grass, Cowhage, Cowslip, Cramp bark, Cranberry, Creatine, Croton Seeds, Cubebs, Cucumber, Cudweed, Cumin, Cursed Buttercup, Cyanostane, Cyclamen, Cypress, D-Mannose, Daffodil, Damiana, Dandelion, Danshen, Date Palm, Datura wrightii, Deanol, Deer Velvet, Delta-8-Tetrahydrocannabinol (Delta-8-THC), Delta-9-Tetrahydrocannabinol (THC), Dendrobium, Desert Parsley, Devil's Claw, Devil's Club, DHEA, Diacylglycerol, Diatomaceous earth, Diindolylmethane, Diiodothyronine, Dill, Dimethylglycine (DMG), Dimethylhexylamine (DMHA), Dimethylsulfoxide (DMSO), Diosmin, Divi-Divi, Docosahexaenoic Acid (DHA), Dodder, Dolomite, Dong Quai, Douglas fir, Dragon Fruit, Dragon's Blood, Duckweed, Dulse, Durabolin, Durian, Dusty Miller, Dwarf Elder, Dwarf Pine Needle, Dyer's Broom, Dymethazine, Eastern Hemlock, Eastern Red Cedar, Ecdysteroids, Echinacea, Ecklonia Cava, Eicosapentaenoic Acid (EPA), Elderberry, Elderflower, Elecampane, Elemi, Eleuthero, Ellagic Acid, Elm Bark, Emu Oil, English Adder's Tongue, English Horsemint, English Ivy, English Walnut, Ephedra, Epiandrosterone, Epistane, Equol, Ergot, Ergothioneine, Eryngo, Eucalyptus, Euphorbia cyparissias, Euphorbia hirta, European Barberry, European Buckthorn, European Chestnut, European Five-finger Grass, European Mandrake, European Mistletoe, Eurycoma longifolia, Evening Primrose, Evodia, Eyebright, Fadogia Agrestis, False Unicorn, Fennel, Fenugreek, Fermented Milk, Fermented Wheat Germ Extract, Fever Bark, Feverfew, Ficin, Field Scabious, Fig, Figwort, Fir, Fireweed, Fish Oil, Flaxseed, Flaxseed Oil, Fluoride, Fly Agaric Mushroom, Fo-Ti, Folic Acid, Fool's Parsley, Forget-Me-Not, Forsythia, Foxglove, Frankincense, Fringetree, Frostwort, Fructo-Oligosaccharides (FOS), Fucus Vesiculosus, Fulvic Acid, Fumitory, Galacto-oligosaccharides (GOS), Galbanum, Galphimia Glauca, Gamboge, Gamma Butyrolactone (GBL), Gamma-Aminobutyric Acid (GABA), Gamma-Hydroxybutyrate (GHB), Gamma-Linolenic Acid (GLA), Gamma-oryzanol, Garcinia, Garden Cress, Gardenia, Garlic, Gelatin, Gelsemium, Genistein Combined Polysaccharide, Gentian, German Chamomile, German Ipecac, German Sarsaparilla, Germander, Germanium, Ginger, Ginkgo, Globe Flower, Globemallow, Glossy Privet, Glucomannan, Glucosamine, Glucuronolactone, Glutaurine, Glutathione, Glycerol, Glycolic Acid, Glycomacropeptide, Glyconutrients, Goa Powder, Goat's Rue, Goji, Golden Ragwort, Goldenrod, Goldenseal, Goldthread, Gossypol, Gotu Kola, Goutweed, Grains of Paradise, Grape, Grapefruit, Gravel Root, Graviola, Great Plantain, Greater Burnet, Greater Celandine, Greek Sage, Green Coffee, Green Tea, Griffonia simplicifolia, Ground Ivy, Ground Pine, Groundsel, Guaiac Wood, Guar Gum, Guarana, Guarumo, Guava, Guggul, Gum arabic, Gumweed, Gymnema, Halodrol-50, Haronga, Hartstongue, Hawaiian baby woodrose, Hawthorn, Hazelnut, Heart's Ease, Heather, Hedge Mustard, Hedge-Hyssop, Hemlock, Hemlock Water Dropwort, Hemp, Hemp Agrimony, Hempnettle, Henbane, Henna, Herb Paris, Herb Robert, Hercules Club, Hesperidin, Hexadrone, Hexylamine, Hibiscus sabdariffa, Higenamine, Holly, Hollyhock, Holy Basil, Homotaurine, Honey, Honeysuckle, Hoodia, Hops, Hordenine, Horny Goat Weed, Horse Chestnut, Horsemint, Horseradish, Horsetail, Hound's Tongue, Houseleek, Hu Zhang, Humic Acid, Huperzine A, Hyacinth Bean, Hyaluronic Acid, Hydrangea, Hydrazine Sulfate, Hydroxymethylbutyrate (HMB), Hyperimmune Egg, Hyssop, Iboga, Iceland Moss, Idebenone, Ignatius Bean, Immortelle, Indian Cassia, Indian Gooseberry, Indian Long Pepper, Indian Snakeroot, Indigo pulchra, Indium, Indole-3-Carbinol, Inosine, Inositol, Inositol Nicotinate, Inulin, Iodine, IP-6 (inositol hexaphosphate), Ipecac, Iporuru, Ipriflavone, Iron, Irvingia gabonensis, Isatis, Isopropylnorsynephrine, Ivy Gourd, Jaborandi, Jackfruit, Jalap, Jamaican Dogwood, Jambolan, Japanese Apricot, Japanese Mint, Japanese Persimmon, Jasmine, Java Tea, Javanese Turmeric, Jenny Craig Diet, Jequirity, Jewelweed, Jiaogulan, Jimson Weed, Job's Tears, Jojoba, Juniper, Justicia pectoralis, K2/Spice, Kale, Kamala, Kaolin, Karaya Gum, Kava, Kefir, Ketogenic diet, Khat, Khella, Kinetin, Kiwi, Knotweed, Kohlrabi, Kombucha, Korean Pine, Kousso, Kratom, Krill Oil, Kudzu, Labdanum, Laburnum, Lactase, Lactic Acid, Lacticaseibacillus casei, Lacticaseibacillus paracasei, Lacticaseibacillus rhamnosus, Lactiplantibacillus pentosus, Lactiplantibacillus plantarum, Lactobacillus acidophilus, Lactobacillus crispatus, Lactobacillus delbrueckii, Lactobacillus gasseri, Lactobacillus helveticus, Lactobacillus jensenii, Lactobacillus johnsonii, Lactoferrin, Lady's Bedstraw, Laminaria, Larch Arabinogalactan, Larch Turpentine, Lathyrus, Latilactobacillus sakei, Laurelwood, Lauric Acid, Lavender, Lavender Cotton, Laxogenin, Lecithin, Lemon, Lemon Balm, Lemon Eucalyptus, Lemon Verbena, Lemongrass, Lentinan, Lesser Celandine, Levant Berry, Levilactobacillus brevis, Licorice, Ligandrol, Ligilactobacillus salivarius, Lily-of-the-Valley, Lime, Limonene, Limosilactobacillus fermentum, Limosilactobacillus reuteri, Linden, Lingonberry, Lion's Mane Mushroom, Lipase, Lithium, Liver Extract, Liverwort, Lobelia, Logwood, Lorenzo's Oil, Lotus, Lousewort, Lovage, Luffa, Lunasin, Lungmoss, Lungwort, Lupin, Lutein, Lychee, Lycopene, M1-4ADD (17a-methyl-androsta-1,4-diene-3b), Maca, Macadamia Nut, Mace, Madagascar Periwinkle, Madder, Magnesium, Magnolia, Maidenhair Fern, Maitake Mushroom, Malabar Nut, Male Fern, Malic Acid, Mallow, Manaca, Manchurian Thorn, Manganese, Mangosteen, Manna, Maqui, Maral Root, Maritime Pine, Marjoram, Marsh Blazing Star, Marsh Labrador Tea, Marsh Marigold, Marshmallow, Masterwort, Mastic, Meadowsweet, Medium Chain Triglycerides (MCTs), Melanotan, Melatonin, Mentabolan, Mercury Herb, Mesoglycan, Methasterone, Methoxydienone, Methoxylated Flavones, Methyldiazinol, Methylstenbolone, Methylsulfonylmethane (MSM), Methylsynephrine, Mezereon, Milk Thistle, Miracle Fruit, Molybdenum, Moneywort, Monolaurin, Monterey Pine, Moringa, Mormon Tea, Motherwort, Mountain Ash, Mountain Flax, Mountain Laurel, Mouse-Ear Hawkweed, Mugwort, Muira Puama, Mullein, Musk, Myrcia, Myrrh, Myrtle, N, N-DMPEA (N,N-Dimethylphenethylamine), N-Acetyl Cysteine (NAC), N-Methyltyramine, NADH, Nasturtium, Nattokinase, Neem, Nerve Root, New Jersey Tea, New Zealand Green-Lipped Mussel, Niacin, Niacinamide, Nicotinamide Mononucleotide, Niauli oil, Nickel, Nicotinamide Riboside, Nikko Maple, Noni, Northern Prickly Ash, Norway Spruce, Nutmeg, Nux Vomica, Oak Moss, Oats, Octacosanol, Octopamine, Oleander, Oleic Acid, Olive, Olive Oil, Omega-6 Fatty Acids, Onion, Oolong Tea, Orchic extract, Oregano, Oregon Grape, Oriental Arborvitae, Ornamental Marigold, Ornithine, Ornithine Ketoglutarate (OKG), Orris, Oscillococcinum, Osha, Ostarine, Ostrich Fern, Oswego Tea, Ox-Eye Daisy, Padang Cassia, Pagoda Tree, Palm Oil, Palmitoylethanolamide (PEA), Panax ginseng, Panax notoginseng, Pancreatic Enzyme Products, Pangamic Acid, Pantethine, Pantothenic Acid, Pao Pereira, Papain, Papaya, Para-Aminobenzoic Acid (PABA), Pareira, Parsley, Parsley Piert, Parsnip, Partridgeberry, Passion Flower, Pata De Vaca, Patchouli Oil, Pau D'Arco, Pea Protein, Peanut Oil, Pear, Pectin, Pedunculate Oak, Pellitory, Pellitory-of-the-Wall, Pennyroyal, Peony, Peppermint, Perilla, Perillyl Alcohol, Periwinkle, Peru Balsam, Peyote, Phaseolus vulgaris, Pheasant's Eye, Phellodendron, Phenethylamine (PEA), Phenibut, Phenpromethamine, Phlorizin, Phosphate Salts, Phosphatidylcholine, Phosphatidylserine, Phytase, Picamilon, Picrorhiza, Pimpinella, Pinellia Ternata, Pink Root, Pipsissewa, Piracetam, Pitcher Plant, Plant Sterols, Pleurisy root, Plum, Podophyllum, Poinsettia, Poison Ivy, Pokeweed, Polarity Therapy, Policosanol, Polydextrose, Polypodium Leucotomos, Pomegranate, Poplar, Poppy Seed, Poria Mushroom, Potassium, Potato, Potentilla, Pregnenolone, Premorse, Prickly Pear Cactus, Procaine, Progesterone, Propolis, Proteolytic Enzymes (Proteases), Psilocybin, Pu-erh Tea, Pulsatilla, Pumpkin, Purple Loosestrife, Purple Nut Sedge, Pygeum, Pyrethrum, Pyruvate, Quassia, Quebracho Blanco, Queen's Delight, Quercetin, Quillaia, Quince, Quinoa, Rabdosia Rubescens, Radish, Raspberry Ketone, Rauvolfia Vomitoria, Rauwolscine, Red Clover, Red Maple, Red Raspberry, Red Sandalwood, Red Soapwort, Red Yeast Rice, Red-spur Valerian, Reed Herb, Rehmannia, Reishi Mushroom, Resveratrol, Rhatany, Rhodiola, Rhubarb, Riboflavin, Ribose, Rice Bran, Rice Bran Arabinoxylan Compound, Rice protein, RNA and DNA, Rock Rose, Roman Chamomile, Rooibos, Rose Geranium Oil, Rose Hip, Rosemary, Royal Jelly, Rue, Rupturewort, Rusty-leaved Rhododendron, Rutin, Rye Grass, S-23, Saccharomyces boulardii, Safed Musli, Safflower, Saffron, Sage, Saigon Cinnamon, Salacia, Salatrim, Salep, Salvia divinorum, SAMe, Samphire, Sandy Everlasting, Sangre de Grado, Sanicle, Sarsaparilla, Sassafras, Savin Tops, Saw Palmetto, Scarlet Pimpernel, Sceletium, Schisandra, Schizonepeta, Scopolia, Scotch Broom, Scotch Thistle, Scurvy Grass, Sea Buckthorn, Sea Moss, Secretin, Securinega Suffruticosa, Selenium, Self-Heal, Senega, Senna, Serrapeptase, Sesame, Sessile Oak, Shark Cartilage, Shark Liver Oil, Shea Butter, Shellac, Shepherd's Purse, Shiitake Mushroom, Siberian Cocklebur, Sida cordifolia, Silicon, Simaruba, Sitostanol, Skullcap, Skunk Cabbage, Slippery Elm, Smartweed, Smooth Alder, Snake Skin, Sodium, Sodium Bicarbonate, Sodium Tetrachloroaurate, Solomon's Seal, Sorghum, Sorrel, Soy, Soybean Oil, Spanish Broom, Spanish Origanum Oil, Spearmint, Spinach, Spiny Restharrow, Spleen extract, Spotted Geranium, Squalamine, Squill, St. John's Wort, Star Anise, Star of Bethlehem, Stavesacre, Stenabolic, Stereosp ermum, Stevia, Stinging Nettle, Stone Root, Storax, Strawberry, Streptococcus thermophilus, Strontium, Strophanthus, Succinate, Sulbutiamine, Sulforaphane, Sulfur, Suma, Sumbul, Summer Savory, Sundew, Sunflower Oil, Superoxide Dismutase (SOD), Swallowroot, Swamp Milkweed, Sweet Almond, Sweet Annie, Sweet Cherry, Sweet Cicely, Sweet Clover, Sweet Gale, Sweet Orange, Sweet Sumac, Sweet Vernal Grass, Sweet Violet, Sweet Woodruff, Syrian Rue, Tamarind, Tamarix dioica, Tangerine, Tannic acid, Tansy, Tansy Ragwort, Tapioca, Tarragon, Tart Cherry, Tartaric Acid, Taurine, Tea Tree Oil, Teazle, Terminalia, Testolone, Tetrahydrocannabivarin (THCV), Theacrine, Theaflavin, Theanine, Thiamine, Thuja, Thunder God Vine, Thyme, Thymus extract, Thyroid Extract, Tianeptine, Timothy Grass, Tin, Tinospora cordifolia, Tiratricol, Tocotrienols, Tolu Balsam, Tomato, Tonka Bean, Toothed Clubmoss, Tormentil, Tragacanth, Trailing Arbutus, Transfer Factor, Traveler's Joy, Tree of Heaven, Tree Tobacco, Tree Turmeric, Trendione, Tribulus, Trichopus zeylanicus, Tronadora, Trypsin, Tung Seed, Turkey Corn, Turkey Tail Mushroom, Turmeric, Turpentine Oil, Turtlehead, Tylophora, Tyramine, Ubiquinone, Umckaloabo, Usnea, Uva ursi, Uzara, Valerian, Vanadium, Vanilla, Verbena, Veronica, Vetiver, Vietnamese Coriander, Vinpocetine, Vitamin A, Vitamin B1, Vitamin B2, Vitamin B12, Vitamin B6, Vitamin C, Vitamin D, Vitamin E, Vitamin K, Vitamin O, Wafer Ash, Wahoo, Wallflower, Wasabi, Water Avens, Water Dock, Water Hemlock, Watercress, Wheat Bran, Wheatgrass, Whey Protein, White Dead Nettle Flower, White Hellebore, White Horehound, White Lily, White Mulberry, White Mustard, White Oak, White Pepper, White Sandalwood, Wild Carrot, Wild Cherry, Wild Daisy, Wild Indigo, Wild Lettuce, Wild Mint, Wild Thyme, Wild Yam, Willard Water, Willow Bark, Wine, Winter Cherry, Winter Savory, Wintergreen, Witch Hazel, Wood Anemone, Wood Sage, Wood Sorrel, Wormseed, Wormwood, Xanthan Gum, Xanthoparmelia, Xylitol, Yarrow, Yellow Dock, Yellow Loosestrife, Yellow Toadflax, Yerba Mansa, Yerba Mate, Yerba Santa, Yew, Yin Chen, Ylang Ylang Oil, Yogurt, Yohimbe, Yucca, Zeaxanthin, Zedoary, Zinc, Zizyphus, alpha lipoic acid, aniracetam, phenylpiracetam, Bacopa monnieri, Celastris paniculatus, centrophenoxine, Clitoria ternatea, coluracetam, Convolvulus pluricaulis, ubiquinone (for example, Coenzyme Q10), creatine or a salt, ester, or amide suitable for human ingestion thereof, choline or a salt, ester, or amide suitable for human ingestion thereof, dimethylethanolamine (DMAE), forskolin, Ginkgo biloba, ginseng, kanna, kava kava, lecithin or a salt, ester, or amide suitable for human ingestion thereof, medium chain triglycerides, reduced nicotinamide adenine dinucleotide (NADH), nefiracetam, nicotine, arecoline, noopept, oatstraw, oxiracetam, passion flower, pine bark extract, pramiracetam, pyrroloquinoline quinone (PQQ), pterostilbene, Rhodiola rosea, S-adenosyl methionine (SAMe), schizandrol-A, taurine or a salt, ester, or amide suitable for human ingestion thereof, L-tyrosine or a salt, ester, or amide suitable for human ingestion thereof, N-acetyl-L-tyrosine or a salt, ester, or amide suitable for human ingestion thereof, L-glutamine or a salt, ester, or amide suitable for human ingestion thereof, alanine or a salt, ester, or amide suitable for human ingestion thereof, beta-alanine or a salt, ester, or amide suitable for human ingestion thereof, phenylalanine (such as L-phenylalanine) or a salt, ester, or amide suitable for human ingestion thereof, L-theanine or a salt, ester, or amide suitable for human ingestion thereof, L-tryptophan or a salt, ester, or amide suitable for human ingestion thereof, L-arginine or a salt, ester, or amide suitable for human ingestion thereof, L-carnitine or a salt, ester, or amide suitable for human ingestion thereof, L-citrulline or a salt, ester, or amide suitable for human ingestion thereof, L-cysteine or a salt, ester, or amide suitable for human ingestion thereof, L-ornithine or a salt, ester, or amide suitable for human ingestion thereof, L-aspartate or a salt, ester, or amide suitable for human ingestion thereof, glutamine or a salt, ester, or amide suitable for human ingestion thereof, glycine or a salt, ester, or amide suitable for human ingestion thereof, histidine or a salt, ester, or amide suitable for human ingestion thereof, lysine or a salt, ester, or amide suitable for human ingestion thereof, methionine or a salt, ester, or amide suitable for human ingestion thereof, proline or a salt, ester, or amide suitable for human ingestion thereof, serine or a salt, ester, or amide suitable for human ingestion thereof, threonine or a salt, ester, or amide suitable for human ingestion thereof, tyrosine or a salt, ester, or amide suitable for human ingestion thereof, carnitine or a salt, ester, or amide suitable for human ingestion thereof, acetyl-L-carnitine or a salt, ester, or amide suitable for human ingestion thereof, propionyl-L-carnitine or a salt, ester, or amide suitable for human ingestion thereof, uridine, thiamine or a salt, ester, or amide a salt, ester, or amide suitable for human ingestion thereof, niacin or a salt, ester, or amide suitable for human ingestion thereof, pantothenic acid or a salt, ester, or amide suitable for human ingestion thereof, or a salt, ester, or amide suitable for human ingestion thereof, vitamin B8 or a salt, ester, or amide suitable for human ingestion thereof, vitamin B9 or a salt, ester, or amide suitable for human ingestion thereof, vitamin B12 or a salt, ester, or amide suitable for human ingestion thereof.

Compositions and/or formulations of the present disclosure may be administered in any form, including a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof for example. Compositions and/or formulations of the present disclosure may also include an acceptable additive (e.g., one of a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof) and/or an acceptable carrier (e.g., one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). Also said compositions may be used to readily prepare injectable formulations containing water-soluble metal-capsaicinoid complexes, by for example diluting them with water for injection.

Implementations of the water-soluble metal-capsaicinoid complexes may conveniently be presented in unit dosage form. Unit dosage formulations may be those containing a daily dose or unit, a daily sub-dose, or an appropriate fraction thereof, of the administered components as described herein.

A dosage unit may include the water-soluble metal-capsaicinoid complexes or their derivatives and metabolites and at least one pharmaceutically acceptable additive. The additive may be, but not limited to, a sugar or a casein containing compound such as casein powder, to prevent gastrointestinal discomfort upon ingestion.

The dosage for oral use of the water-soluble metal-capsaicinoid complexes can be from 0.1 to 1000 mg per day, split into one or more dosages. For exercise performance enhancing purposes, the formulation is administered (for example, by ingestion) prior to exercise, for example any time between 3 days prior to exercise and right before exercise. In certain implementations, the formulation is administered half an hour before exercise.

The dosage units may be in a form suitable for administration by standard routes. In general, the dosage units may be administered, by non-limiting example, by the oral (including buccal and sublingual), transdermal, topical, rectal, ophthalmic (including intravitreal or intracameral), nasal, vaginal, and/or parenteral (including subcutaneous, intramuscular, intravenous, intradermal, intratracheal, and epidural) routes.

For the exemplary purposes of this disclosure, oral delivery may be a particularly advantageous delivery route for administration to humans and animals of implementations of a pharmaceutical composition, optionally formulated with appropriate pharmaceutically acceptable additives to facilitate administration.

It should be appreciated that any of the components of particular implementations of the metal complexes of capsaicinoids may be used as supplied commercially, or may be preprocessed by, by non-limiting example, any of the methods and techniques of agglomeration, air suspension chilling, air suspension drying, balling, coacervation, comminution, compression, pelletization, cryopelletization, extrusion, granulation, homogenization, inclusion compoundation, lyophilization, melting, mixed, molding, pan coating, solvent dehydration, sonication, spheronization, spray chilling, spray congealing, spray drying, and/or other processes known in the art depending in part on the dosage form desired. The various components may also be pre-coated or encapsulated as known in the art. It will also be clear to one of ordinary skill in the art that appropriate additives may also be introduced to the composition or during the processes to facilitate the preparation of the dosage forms, depending on the need of the individual process.

Those of ordinary skill in the art will be able to readily select manufacturing equipment and pharmaceutically acceptable additives or inert ingredients to manufacture implementations of the water-soluble metal-capsaicinoid complexes. For the exemplary purposes of this disclosure, some examples of pharmaceutically acceptable additives or inert ingredients and manufacturing process are included below, particularly those that relate to manufacture of implementations of the water-soluble metal-capsaicinoid complexes in tablet form. Notwithstanding the specific examples given, it will be understood that those of ordinary skill in the art will readily appreciate how to manufacture implementations of the water-soluble metal-capsaicinoid complexes according to the other methods of administration and delivery disclosed in this document.

A particular implementation of the water-soluble metal-capsaicinoid complexes may include a lubricant. Lubricants are any anti-sticking agents, glidants, flow promoters, and the like materials that perform a number of functions in tablet manufacture, for example, such as improving the rate of flow of the tablet granulation, preventing adhesion of the tablet material to the surface of the dies and punches, reducing interparticle friction, and facilitating the ejection of the tablets from the die cavity. Lubricants may comprise, for example, magnesium stearate, calcium stearate, talc, and colloidal silica.

Particular implementations of the water-soluble metal-capsaicinoid complexes may also include a binder. Binders are any agents used to impart cohesive qualities to powdered material through particle-particle bonding. Binders may include, for example, matrix binders (e.g., dry starch, dry sugars), film binders (e.g., celluloses, bentonite, sucrose), and chemical binders (e.g., polymeric cellulose derivatives, such as methyl cellulose, carboxy methyl cellulose, and hydroxy propyl cellulose); and other sugar, gelatin, non-cellulosic binders and the like.

Disintegrators may be used in particular implementations of the water-soluble metal-capsaicinoid complexes to facilitate the breakup or disintegration of tablets after administration. Disintegrators may include, for example, starch, starch derivatives, clays (e.g., bentonite), algins, gums (e.g., guar gum), cellulose, cellulose derivatives (e.g., methyl cellulose, carboxymethyl cellulose), croscarmellose sodium, croscarmellose cellulose, and other organic and inorganic materials.

Implementations of the water-soluble metal-capsaicinoid complexes may include diluents, or any inert substances added to increase the bulk of the water-soluble metal-capsaicinoid complexes, such as potassium capsaicin complex to make a tablet a practical size for compression. Diluents may include, but are not limited to, for example, calcium phosphate, calcium sulfate, lactose, mannitol, magnesium stearate, potassium chloride, and citric acid, among other organic and inorganic materials.

Buffering agents may be included in a metal-capsaicinoid complexs and may be any one of an acid and a base, where the acid is, but not limited to, for example, citric acid, propionic acid, p-toluenesulfonic acid, salicylic acid, stearic acid, succinic acid, tannic acid, tartaric acid, thioglycolic acid, or toluenesulfonic acid, and the base is, for example, ammonium hydroxide, potassium hydroxide, sodium hydroxide, sodium hydrogen carbonate, aluminum hydroxide, calcium carbonate, and other organic and inorganic chemicals.

Implementations of the water-soluble metal-capsaicinoid complexes or their derivatives and metabolites may also be administered through use of amphipathic lipid delivery systems (such as liposomes and unilamellar vesicles), caplet systems, oral liquid systems, parenteral and/or intravenous systems, topical systems (creams, gels, transdermal patches, etc.), intranasal systems, rectal or vaginal systems, and many other delivery methods and/or systems known to those of ordinary skill in the art.

Those of ordinary skill in the art will readily be able to select additional pharmaceutically acceptable additives to enable delivery of implementations of a pharmaceutical composition from the disclosure in this document.

With respect to the delivery of particular implementations of the water-soluble metal-capsaicinoid complexes (such as the KOH-capsaicin complex), for the exemplary purposes of this disclosure, tablets may be utilized. Tablets are any solid pharmaceutical dosage form containing a pharmaceutically acceptable active agent or agents to be administered with or without suitable pharmaceutically acceptable additives and prepared either by compression or molding methods well known in the art. Tablets have been in widespread use and remain popular as a dosage form because of the advantages afforded both to the manufacturer (e.g., simplicity and economy of preparation, stability, and convenience in packaging, shipping, and dispensing) and the patient (e.g., accuracy of dosage, compactness, portability, blandness of taste, and ease of administration). Although tablets are most frequently discoid in shape, they may also be, for example, round, oval, oblong, cylindrical, rectangular, or triangular. The tablets may be optionally scored so that they may be separated into different dosages. They may differ greatly in size and weight depending on the amount of the pharmaceutically acceptable active agent or agents present and the intended route of administration. They are divided into two general classes: compressed tablets and molded tablets.

Tablets and other orally discrete dosage forms, such as capsules, cachets, pills, granules, pellets, beads, and particles, for example, may optionally be coated with one or more enteric coatings, seal coatings, film coatings, barrier coatings, compress coatings, fast disintegrating coatings, or enzyme degradable coatings for example. Multiple coatings may be applied for desired performance. Further, dosage forms may be designed for, by non-limiting example, immediate release, pulsatile release, controlled release, extended release, delayed release, targeted release, synchronized release, or targeted delayed release. For release/absorption control, carriers may be made of various component types and levels or thicknesses of coats. Such diverse carriers may be blended in a dosage form to achieve a desired performance. In addition, the dosage form release profile may be affected by a polymeric matrix composition, a coated matrix composition, a multi-particulate composition, a coated multi-particulate composition, an ion-exchange resin-based composition, an osmosis-based composition, or a biodegradable polymeric composition.

While manufacture of implementations of the water-soluble metal-capsaicinoid complexes have been described in particular sequences of steps and/or in particular forms in the examples, it will be understood that such manufacture is not limited to the specific order of steps or forms as disclosed. Compounds of equivalent use/activity may be substituted for the compounds mentioned in the formulation. Any steps or sequences of steps of manufacture of implementations of the water-soluble metal-capsaicinoid complexes in any form are given as examples of possible steps or sequences of steps or potential forms and not as limitations, since many possible manufacturing processes and sequences of steps may be used to manufacture the water-soluble metal-capsaicinoid complexes.

In other aspects, the disclosure relates to methods of treating a disease/condition on a subject. Such diseases and disorders that can be treated by the compounds of the invention include, but are not limited to: acute pain, chronic pain, back pain, diabetic neuropathy, postherpetic neuralgia, migraine, cluster headache, allergic rhinitis, non-allergic rhinitis, osteoarthritis, postoperative nausea and vomiting, burning mouth syndrome, cardiovascular disease, hyperlipidemia, dry skin, dyslipidemia, dyspepsia, fibromyalgia, gastroesophageal reflux disease, HIV related peripheral neuropathy, hypertension, intermetatarsal neuroma, irritable bowel syndrome, laryngitis, motion sickness, myofascial face syndrome, neck pain, neuropathic pain, obesity, peptic ulcers, prurigo nodularis, sinonasal polyposis and swallowing dysfunction. Those diseases and conditions shall be collectively called “capsaicin-treatable conditions”. It may also be used to increase athletic performance, increase fat loss, reduce body fat, decrease blood sugar, increase insulin sensitivity, increase endurance, increase muscular strength, increase absorption of creatine. Those uses shall be referred to as “capsaicin-caused health improvements”. It shall be understood that by describing that the metal-capsaicinoid complex disclosed herein improves any of the above parameters, it means that a subject tested on those parameters would perform better after administration of the metal-capsaicinoid complexes described therein than if they were not administered to the subject.

The method may include administering a metal-capsaicinoid complex such as capsaicin-potassium complex or water-soluble metal-capsaicinoid complexes metabolites to a subject. Thus, use of a metal-capsaicinoid complex such as capsaicin-potassium complex for the treatment of capsaicin-improvable condition is disclosed. In some respects, the use of a metal-capsaicinoid complex such as capsaicin-potassium complex for the manufacture of medicament, therapeutic composition, or nutraceutical composition is disclosed. Due to the higher bioavailability of said compounds, a lesser dose may be used as opposed to regular capsaicinoids, or a greater therapeutical or health-beneficial effect may be achieved compared to administering the same dose of a regular capsaicinoid. Finally, the compounds of the invention may be able to treat diseases and conditions for which regular capsaicinoids are ineffective.

The symptoms and etiology of capsaicin-improvable conditions that can be ameliorated by administration of water-soluble metal-capsaicinoid complexes and the formulations described herein are described in “Handbook of Diseases”, 3rd Edition by Springhouse Corporation, which is fully incorporated by reference herein. Furthermore, the contents of all references, patents, and published patent applications cited throughout this application, if any, are incorporated herein by reference in their entirety for all purposes.

### EXAMPLES

The present disclosure is further illustrated by the following examples that should not be construed as limiting.

### Example 1: Preparation of Capsaicin-Potassium Complex

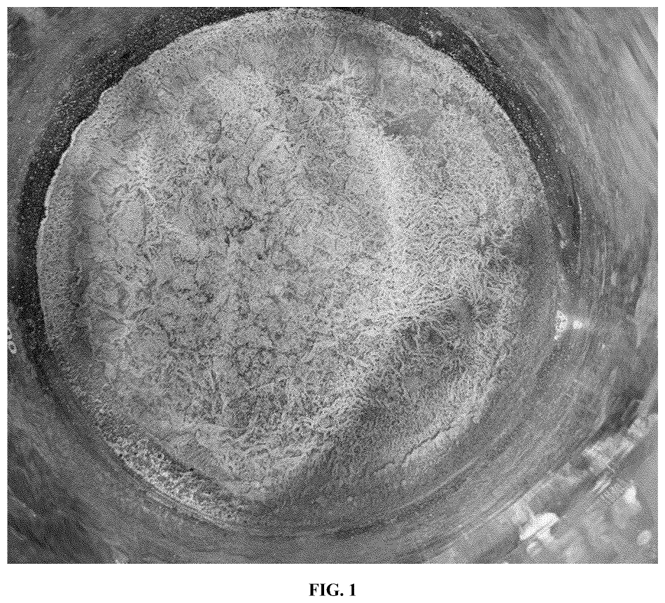

5 grams of potassium hydroxide (anhydrous) were stirred in 100 ml of absolute ethanol in a 1000 ml glass beaker until completely dissolved at room temperature. 5 grams of capsaicin extracted from peppers (which also contains small amounts of other capsaicinoids and natural pigments) were dissolved in ethanol solution containing dissolved potassium hydroxide. The ethanol was evaporated using a stream of hot air which led to the formation of a yellowish to orange solid compound, which can be seen in FIG. 1. The change of color from very light yellow to this orange is possibly due to the complexion reaction.

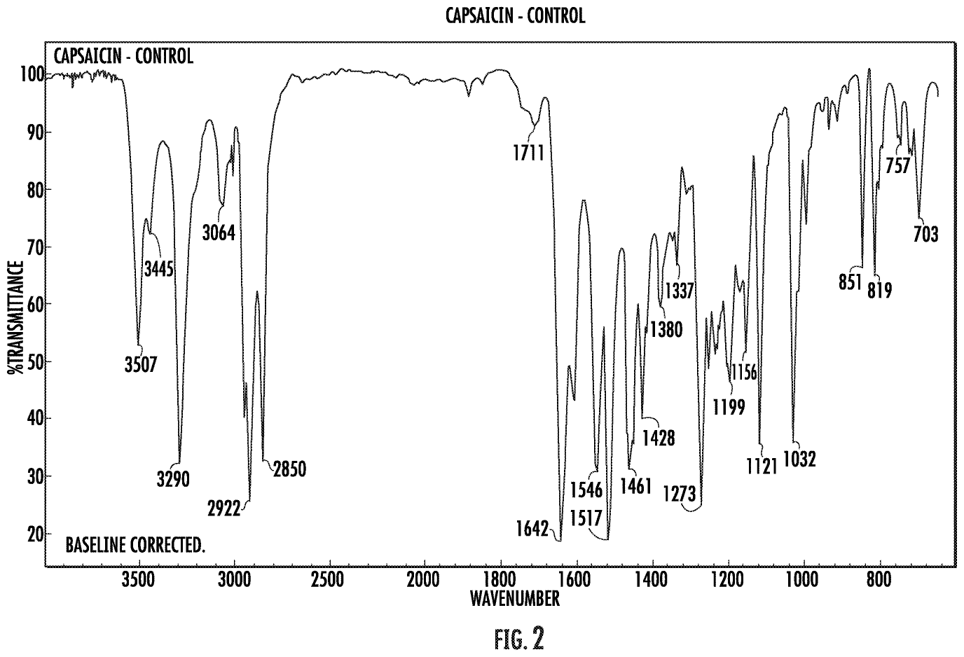

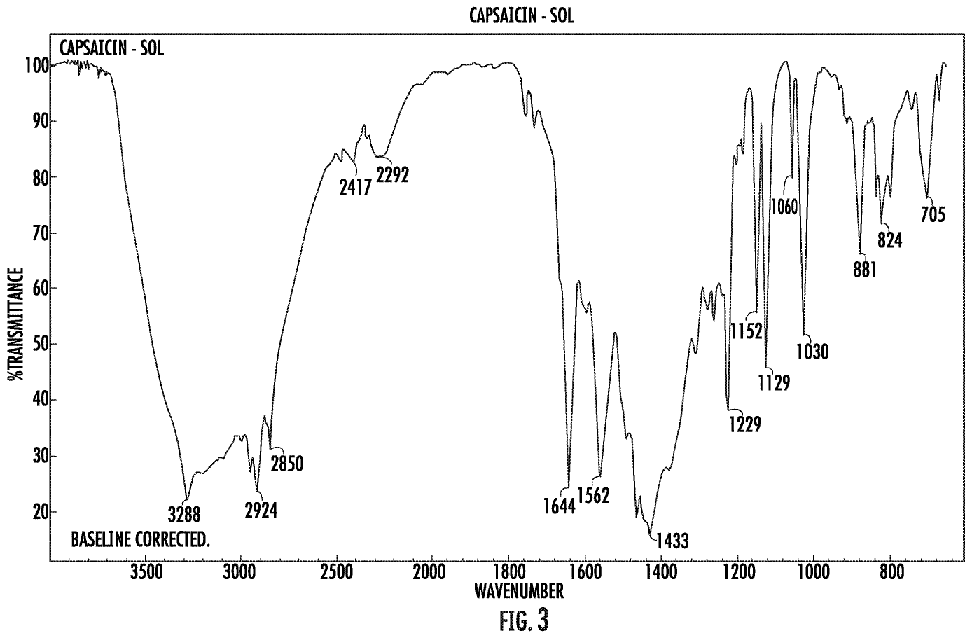

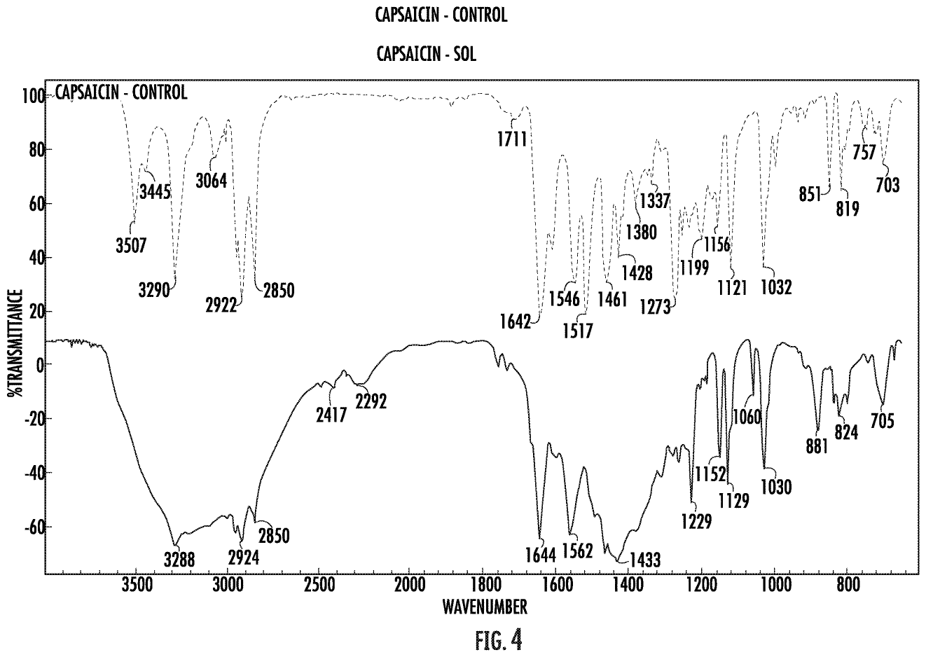

Referring to FIGS. 2-4, Fourier transform infrared spectroscopy confirmed that the resulting yellowish to orange solid compound is a capsaicin-potassium complex:

### Example 2: Solubility and Dissolution Rate of Capsaicin-Potassium Complex

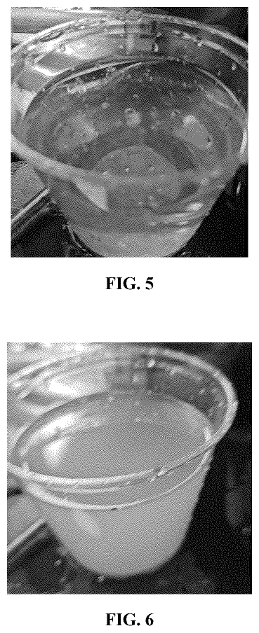

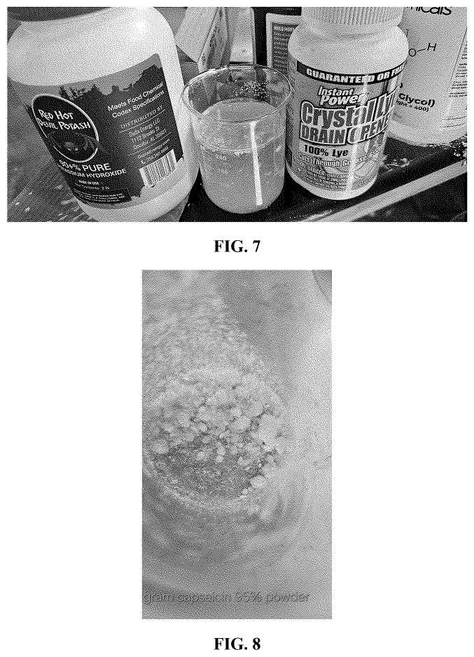

1 gram of the capsaicin-potassium complex produced in Example 1 was put into 100 ml of room temperature water (see FIG. 5). After about 30 seconds of stirring, all the capsaicin dissolved completely into a yellowish solution (see FIG. 6).

The taste, smell, and skin tests (causation of skin burning sensation) confirmed that said dissolved compound was capsaicin.

### Example 3: Solubility of Capsaicin in an Alkaline Medium

In 200 ml of room temperature water, 10 grams of potassium hydroxide and 1 gram of capsaicin were added and stirred for 5 minutes. None of the capsaicin went into solution but remained as aggregates that mostly floated. The situation remained the same after 24 hours.

The experiment was repeated using sodium hydroxide instead of potassium hydroxide (1 gram of capsaicin in ~200 ml of water containing 10 grams sodium hydroxide) and achieved similar results.

### Example 4

In a 1000 ml beaker at room temperature, 50 ml of water, 50 ml of ethanol, and 3 grams of potassium hydroxide were mixed together. 3 grams of capsaicin were then added to the mixture while stirring until dissolved. The liquid mixture was left to dry at 40° C. The resulting solid had a very faint yellow tint (see FIG. 8).

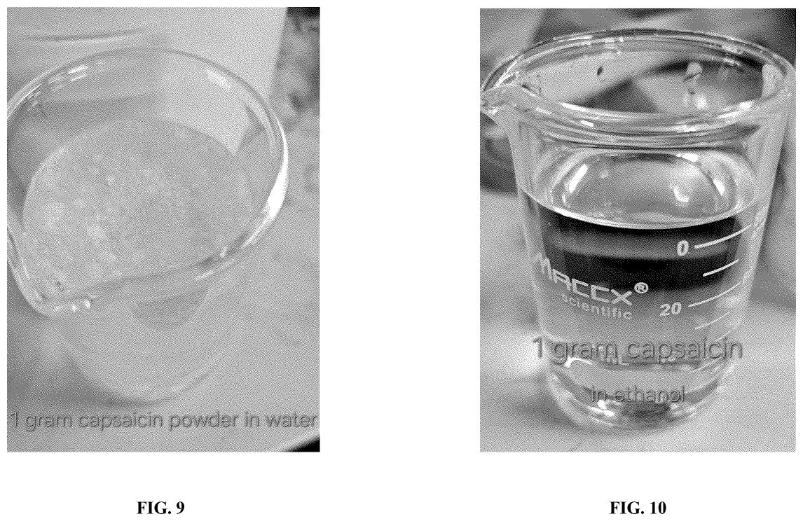

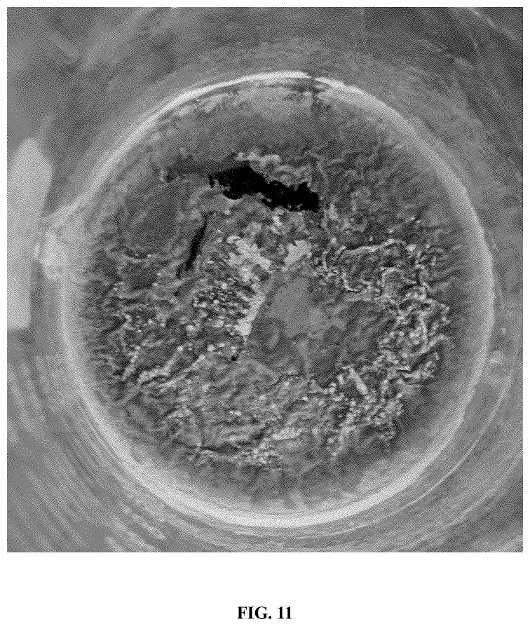

It was not soluble in water (FIG. 9) but retained its solubility in ethanol (FIG. 10).

This could be due to faster evaporation of ethanol than water, aggregation of capsaicin in the aqueous phase, inability of formation of potassium ethoxide, or other reasons. Thus, it is critical for the synthesis methods disclosed herein to be performed in an anhydrous environment (less than 5%, even less than 1%, or even less than 0.1% water).

### Example 5: Preparation of Nonivamide-Potassium Complex

2 grams of potassium hydroxide were dissolved with stirring in 50 ml of absolute ethanol. Any possible water contamination was removed by passing the mixture through a molecular sieve column. 2 grams of nonivamide (Axion labs) were dissolved in the solution. The solution was dried in a 90° C. water bath. The resulting residue was a semi-crystalline solid of yellow to orange color (see FIG. 11).

The semi-crystalline solid has excellent water solubility. 500 mg of this solid dissolves in 100 ml of room temperature water without the need for stirring. It should be noted that previous testing showed that nonivamide, like capsaicin, is not soluble in alkaline aqueous solution.

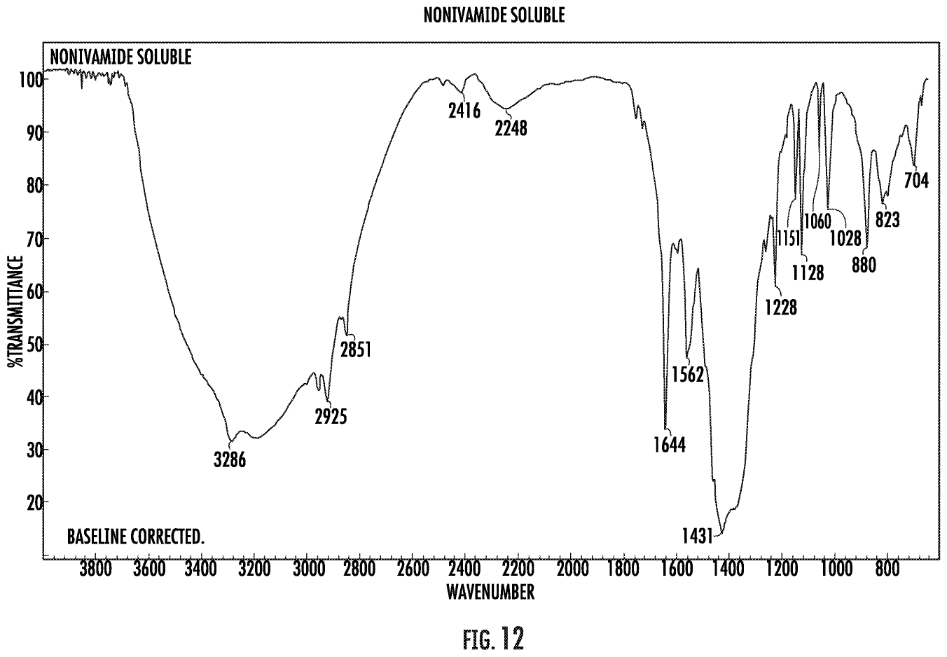

The FTIR spectrum of the nonivamide-potassium complex is shown in FIG. 12.

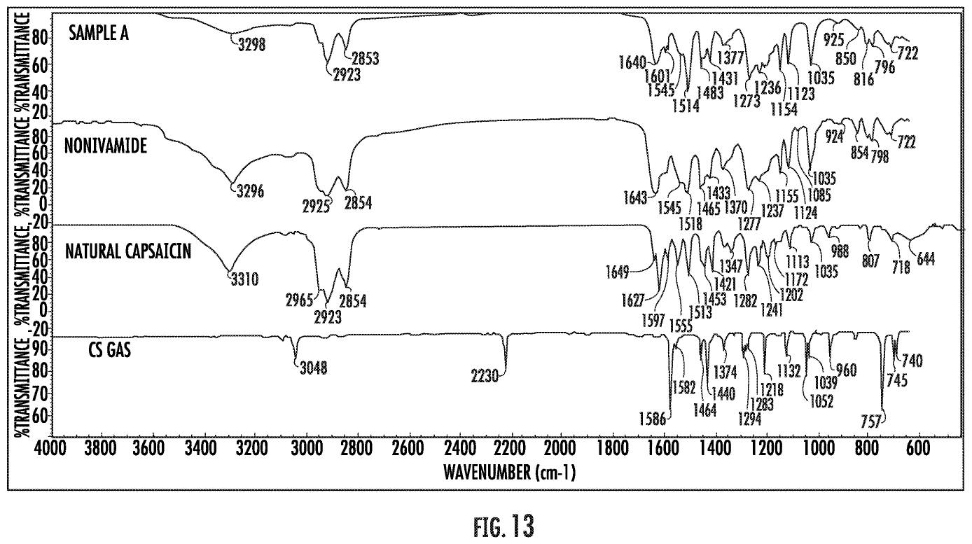

FTIR of nonivamide from Logrado et al., “Counterfeit Pepper Spray: A Case Report.”, Human Factors and Mechanical Engineering for Defense and Safety, 2022, 6: Article 16, is reproduced in FIG. 13.

### Example 6: Activity of Regular Capsaicin Versus Capsaicin-Sodium Complex

20 mg of regular capsaicin were put into a gelatin capsule and ingested by a 43 year old male in the morning on empty stomach (more than 8 hours after ingesting any food) with a glass of water. About 30 minutes later the subject experienced mild stomach discomfort with no notable sweating, diaphoresis, sense of burning, or increase of skin temperature (as measured by a forehead temperature).

The following day the subject ingested 20 mg of a capsaicin-sodium complex produced using the method shown in Example 1.5 minutes after ingestion, (the subject) experienced strong stomach burning and pain sensations, which required drinking 500 ml of a yogurt drink to subside it. His skin, especially on the face, turned red, he started sweating and his temperature on his facial skin increased by 0.5° C.

### REFERENCES CITED AND INCORPORATED HEREIN BY REFERENCE

Bernard et al., “Studies of the toxicological potential of capsinoids: X. Safety assessment and pharmacokinetics of capsinoids in healthy male volunteers after a single oral ingestion of CH-19 Sweet extract.” Int J Toxicol, 2008, 27 (Suppl 3): 137-147
Chaiyasit et al., “Pharmacokinetic and the effect of capsaicin in Capsicum frutescens on decreasing plasma glucose level.” J Med Assoc Thai, 2009, 92:108-113
O'Neill J, Brock C, Olesen A E, Andresen T, Nilsson M, Dickenson A H. “Unravelling the mystery of capsaicin: a tool to understand and treat pain.” Pharmacol Rev., 2012, 64 (4): 939-71
Tran and Hadinoto, “A new solubility enhancement strategy of capsaicin in the form of high-payload submicron capsaicin-chitosan colloidal complex,” Colloids and Surfaces A: Physicochemical and Engineering Aspects, 2017. 520:62-71

1. A solid composition of matter comprising:
5-95% by weight of a capsaicinoid; and
5-95% by weight of a metal.

2. The composition of claim 1 where the composition also comprises 5-95% of an alkoxide.

3. The composition of claim 1, wherein the capsaicinoid is capsaicin.

4. The composition of claim 1, wherein the metal is selected from the group consisting of: lithium, sodium, and potassium.

5. The composition of claim 1, wherein the capsaicinoid in the composition is not aggregated together.

6. The composition of claim 1, wherein at least a portion of the capsaicinoid is complexed to the metal.

7. The composition of claim 1, wherein the composition is soluble in water at a solubility of at least 0.1 g/100 ml water.

8. A method of preparing the compositions of any one of claim 1, said method comprising:
dissolving the metal in a polar solvent to produce a solution comprising the dissolved metal;
dissolving the capsaicinoid in the solution comprising the dissolved metal to produce a complexing solution;
stirring the complexing solution until a homogenous solution is formed; and
evaporating the polar solvent in the homogenous solution, thereby producing a dried product comprising metal-capsaicinoid complex.

9. The method of claim 8, where the capsaicinoid and the metal are in a molar ratio of between 1:1 and 1:100 (capsaicinoid: metal).

10. The method of claim 8, where the polar solvent is ethanol.

11. The method of claim 8, where the metal is potassium and the capsaicinoid is capsaicin.

12. A method of treating a capsaicin-treatable condition by administering to a subject a pharmaceutically effective amount of metal-capsaicinoid complex produced according to the method of claim 8.

13. The method of claim 12, where the capsaicin-treatable condition is one of: acute pain, chronic pain, back pain, diabetic neuropathy, postherpetic neuralgia, migraine, cluster headache, allergic rhinitis, non-allergic rhinitis, osteoarthritis, postoperative nausea and vomiting, burning mouth syndrome, cardiovascular disease, hyperlipidemia, dry skin, dyslipidemia, dyspepsia, fibromyalgia, gastroesophageal reflux disease, HIV related peripheral neuropathy, hypertension, intrametatarsal neuroma, irritable bowel syndrome, laryngitis, motion sickness, myofascial face syndrome, neck pain, neuropathic pain, obesity, peptic ulcers, prurigo nodularis, sinonasal polyposis, and swallowing dysfunction.

14. A method of producing a capsaicin-caused health improvement in a subject comprising administering to the subject a pharmaceutically effective amount of a metal-capsaicinoid complex produced according to the method of claim 8.

15. The method of claim 13, where the capsaicin-caused health improvement is one of: increased athletic performance, increased fat loss, increased caloric expenditure, reduction of body fat, decrease in blood sugar, increase of insulin sensitivity, increase of endurance, increase of absorption of creatine, and increase of muscular strength.

16. The method of claim 12, where the subject is orally administered the metal-capsaicinoid complex produced.

17. The method of claim 16, wherein the pharmaceutically effective amount of the metal-capsaicinoid complex is between 1-1000 mg.

18. A supplemental formulation comprising:
a metal-capsaicinoid complex; and
a second supplement ingredient and/or a pharmaceutically acceptable additive.

19. The supplemental formulation of claim 18 wherein the metal-capsaicinoid comprises capsaicin.

20. The supplemental formulation of claim 18, wherein the metal-capsaicinoid comprises a metal selected from the group consisting of: lithium, sodium, and potassium.
